SEQUENCE LISTING (2 sequences)

SEQ ID NO: 1 - protein, 39 aa, synthetic construct
CFNAWDDKVCEHFAVAGICRIPKWKVRVTTNCARSCEVC

SEQ ID NO: 2 - DNA, 117 bp, synthetic construct
  source 1..117: mol_type = other DNA
tgctttaacgcctgggatgacaaagtttgcgagcacttcgccgtggccggaatatgtagaattccaaaatggaaagtcagggtgacgacgaactgcgccaggtcatgtgaagtttgc

(19) United States
(12) Patent Application Publication
(10) Pub. No.: US 2026/0347408 A1
(43) Pub. Date: Nov. 5, 2026

(54) AMYLOID BETA-SPECIFIC PEPTIDE CBRV1-04369 AND COMPOSITION FOR TREATING ALZHEIMER'S DISEASE COMPRISING SAME

(71) Applicants: GACHON UNIVERSITY OF INDUSTRY-ACADEMIC COOPERATION FOUNDATION, Gyeonggi-do (KR); KOREA INSTITUTE OF OCEAN SCIENCE & TECHNOLOGY, Busan (KR)

(72) Inventors: Seungshic YUM, Busan (KR); Yejin JO, Busan (KR); Hyokyoung WON, Busan (KR); Nayoung LEE, Busan (KR); Seong Soo A. AN, Gyeonggi-do (KR); Kyuhwan SHIM, Seoul (KR); Danyeong KIM, Gyeonggi-do (KR); Keun-A CHUNG, Seoul (KR); Tae-Young HA, Gyeonggi-do (KR); Yeji KIM, Seoul (KR)

(21) Appl. No.: 19/099,850

(22) PCT Filed: Aug. 4, 2023
(86) PCT No.: PCT/KR2023/011509

§ 371 Date: May 7, 2025

(30) Foreign Application Priority Data
Aug. 4, 2022 (KR) 10-2022-0097555

(51) Int. Cl. C07K 14/00; A61K 38/00; A61P 25/28; G01N 33/68

(52) U.S. Cl. CPC C07K 14/001; A61P 25/28; G01N 33/6896; A61K 38/00; G01N 2333/001; G01N 2333/4709; G01N 2800/2821

(57) ABSTRACT

The present invention relates to a peptide capable of directly binding to amyloid beta protein Aβ42, known to be a causative agent of Alzheimer's disease, and a composition for treating and preventing Alzheimer's disease, comprising the peptide. The peptide of the present invention can be used to prevent fibrosis and oligomerization of amyloid beta, and thus can be used for the treatment and prevention of amyloidogenic disease such as Alzheimer's disease. In addition, the peptide of the present invention can be used to detect amyloid beta, and thus can be used for the diagnosis of amyloidogenic diseases such as Alzheimer's disease.

### TECHNICAL FIELD

The present disclosure was conducted with the support of the Ministry of Science and ICT of the Republic of Korea under project number 2021R1A6A1A03038996. The research management institution for the project is the National Research Foundation of Korea (NRF), the research program is “Establishment of Research Infrastructure for Science and Engineering,” and the research project title is “Development of Diagnostic and Therapeutic Technologies for Senile Diseases.” The institution conducting the project is Gachon University, and the research period is from Jun. 1, 2021, to May 31, 2030.

The present disclosure was conducted with the support of the Ministry of Oceans and Fisheries of the Republic of Korea under Project No. 201804303. The research management agency for this project is the Korea Institute of Marine Science & Technology Promotion, the research program is “Post-Genome Multi-Ministry Genome Project”, and the research project title is “Establishment of a Marine Animal Genome Information Analysis and Utilization Base”. The project was carried out by the Korea Institute of Ocean Science and Technology, with a research period from Mar. 1, 2021, to Mar. 31, 2022.

This patent application claims the benefit of and priority to Korean Patent Application No. 10-2022-0097555, which was filed with the Korean Intellectual Property Office on Aug. 4, 2022, the disclosure of which is incorporated herein by reference.

The present disclosure relates to a peptide capable of directly binding to amyloid beta protein AB42, which is known to be a causative agent of Alzheimer's disease, and to a composition including the peptide for the treatment and prevention of Alzheimer's disease.

### BACKGROUND ART

The development of Alzheimer's disease therapeutics by major global pharmaceutical companies has been repeatedly unsuccessful. To date, most Alzheimer's disease treatments have been developed based on antibodies that bind to amyloid beta (AB) protein and tau protein; however, these approaches still face significant limitations.

Amyloid beta (AB) protein undergoes oligomerization, leading to the formation of oligomers, which further aggregate and undergo fibrillization to form fibril structures. There is ongoing debate regarding whether oligomers or fibril forms of amyloid beta protein are responsible for neurotoxicity. Based on current research findings, the prevailing view suggests that oligomers are toxic to cells.

A representative method for screening compound-based drug candidates involves evaluating the inhibition of amyloid beta protein fibrillization. However, because fibrillization of amyloid beta protein is also considered a mechanism that protects against cytotoxicity, it may not be an appropriate screening criterion for drug candidates. Furthermore, inhibiting amyloid beta protein fibrillization may increase the stability of toxic oligomers, posing another challenge in drug development.

Given that amyloid beta oligomers are recognized as the primary toxic agents in Alzheimer's disease, diagnosis and treatment that target same have become increasingly important. High binding affinity to amyloid beta oligomers is necessary for effective therapeutics, and candidates screened for treatment must be non-toxic and easily degradable. From this perspective, naturally derived peptides have advantages over synthetic compounds, as they can be readily degraded in vivo, mitigating the toxicity concerns associated with synthetic compounds. Additionally, unlike antibodies, peptides can be synthesized directly, making them a cost-effective alternative.

### DISCLOSURE OF INVENTION

### Technical Problem

Leading to the present disclosure, intensive and thorough research conducted by the present inventors with the aim of searching for a peptide that binds directly to amyloid beta protein to prevent the fibrillization and oligomerization of amyloid beta protein resulted in the finding that the peptide CBRV1-04369 inhibits both fibrillization and oligomerization of amyloid beta protein, demonstrating therapeutic effects against amyloid-related diseases.

Accordingly, an aspect of the present disclosure is to provide a polypeptide including at least one amino acid sequence selected from the group consisting of SEQ ID NOS: 3, 4, 5, 6, and 7.

Another aspect of the present disclosure is to provide a nucleic acid molecule including a nucleotide sequence encoding a polypeptide including at least one amino acid sequence selected from the group consisting of SEQ ID NOS: 3, 4, 5, 6, and 7.

A further aspect of the present disclosure is to provide a recombinant vector comprising a nucleic acid molecule including a nucleotide sequence encoding for a polypeptide including at least one amino acid sequence selected from the group consisting of SEQ ID NOS: 3, 4, 5, 6, and 7.

Yet another aspect of the present disclosure is to provide a host cell transformed with a recombinant vector comprising a nucleic acid molecule including a nucleotide sequence encoding for a polypeptide including at least one amino acid sequence selected from the group consisting of SEQ ID NOS: 3, 4, 5, 6, and 7. Still another aspect of the present disclosure is to provide a pharmaceutical composition for the prevention or treatment of amyloid beta-related neurodegenerative diseases, containing a polypeptide including at least one amino acid sequence selected from the group consisting of SEQ ID NOS: 3, 4, 5, 6, and 7.

Yet still another aspect of the present disclosure is to provide a composition for detecting amyloid beta protein, containing a polypeptide including at least one amino acid sequence selected from the group consisting of SEQ ID NOS: 3, 4, 5, 6, and 7.

A still further aspect of the present disclosure is to provide a method for detecting amyloid beta protein, the method including the steps of contacting a composition with a sample, the composition containing a polypeptide including at least one amino acid sequence selected from the group consisting of SEQ ID NOS: 3, 4, 5, 6, and 7 for detecting amyloid beta protein; and detecting a peptide-amyloid beta protein complex thus formed in the sample.

### Solution to Problem

The present disclosure provides the invention as set forth in the following items 1 to 20.
1. A polypeptide, including at least one amino acid sequence selected from the group consisting of SEQ ID NOS: 3, 4, 5, 6, and 7.
2. The polypeptide of item 1, wherein the polypeptide includes the amino acid sequence of SEQ ID NO: 1.
3. A nucleic acid molecule including a nucleotide sequence encoding the polypeptide of item 1.
4. The nucleic acid molecule of item 3, wherein the nucleotide sequence is the nucleotide sequence of SEQ ID NO: 2.
5. A recombinant vector comprising the nucleic acid molecule of item 3.
6. A host cell transformed with the recombinant vector of item 5.
7. A pharmaceutical composition for prevention or treatment of amyloid beta-related neurodegenerative diseases, the composition containing a polypeptide including at least one amino acid sequence selected from the group consisting of SEQ ID NOS: 3, 4, 5, 6, and 7.
8. The pharmaceutical composition of item 7, wherein the polypeptide includes the amino acid sequence of SEQ ID NO: 1.
9. The pharmaceutical composition of item 7, wherein the polypeptide inhibits fibrillization and oligomerization of amyloid beta.
10. The pharmaceutical composition of item 7, wherein the neurodegenerative disease is selected from the group consisting of Alzheimer's disease, amyloid angiopathy, amyloid-related stroke, systemic amyloidosis, and Parkinson's disease.
11. A composition for detecting amyloid beta protein, comprising a polypeptide including at least one amino acid sequence selected from the group consisting of SEQ ID NOS: 3, 4, 5, 6, and 7.
12. The composition of item 11, wherein the polypeptide includes the amino acid sequence of SEQ ID NO: 1.
13. A method for detecting amyloid beta protein, the method comprising a step of detecting a peptide-amyloid beta protein complex formed by contacting the composition of item 11 with a sample.
14. A method for preventing or treating amyloid beta-related neurodegenerative disease, the method comprising a step of administering the polypeptide of item 1 or 2 or the composition of any one of items 7 to 10 to a subject.
15. Use of a polypeptide comprising at least one amino acid sequence selected from the group consisting of SEQ ID NOS: 3, 4, 5, 6, and 7 for prevention or treatment of amyloid beta-related neurodegenerative disease.
16. The use of item 15 wherein the polypeptide comprises the amino acid sequence of SEQ ID NO: 1.
17. Use of a polypeptide comprising at least one amino acid sequence selected from the group consisting of SEQ ID NOS: 3, 4, 5, 6, and 7 for manufacture of a medicament for preventing or treating amyloid beta-related neurodegenerative disease.
18. The use of item 17, wherein the polypeptide comprises the amino acid sequence of SEQ ID NO: 1 for manufacture of a medicament for preventing or treating amyloid beta-related neurodegenerative disease.
19. A kit for detecting amyloid beta protein, comprising the composition of item 11.
20. A method for diagnosing amyloid beta-related neurodegenerative diseases, the method comprising a step of detecting a peptide-amyloid beta protein complex formed by contacting the polypeptide of item 1 or 2 or the composition of item 11 with a sample.

According to one aspect thereof, the present disclosure provides a polypeptide comprising at least one amino acid selected from the group consisting of SEQ ID NOS: 3, 4, 5, 6, and 7.

In an embodiment of the present disclosure, the peptide includes the amino acid sequence of SEQ ID NO: 1.

In an embodiment of the present disclosure, the peptide consisting of the amino acid sequence of SEQ ID NO: 1 is CBRV1-04369.

Herein, the polypeptide may include at least one additional amino acid at the C-terminal and/or N-terminal thereof. Such additional amino acid residues may be added individually or collectively with the aim of improving production efficiency, purification, stabilization in vivo or in vitro, and coupling or detection of a complex. For instance, the polypeptide may further include a cysteine residue at the C-terminal and/or N-terminal thereof. The additional amino acid residues may also provide a “tag” for the purification or detection of the polypeptide. Such tags may facilitate interactions with specific antibodies, including His6 tags for immobilized metal affinity chromatography (IMAC), His6 tags, (HisGlu) 3 tags (“HEHEHE” tag), “myc” (c-myc) tags, or “FLAG” tags.

The polypeptide comprising at least one selected from the group consisting of SEQ ID NOS: 3, 4, 5, 6, and 7 of the present disclosure is interpreted to include not only amino acid sequences selected from the group consisting of SEQ ID NOS: 3, 4, 5, 6, and 7 but also sequences that exhibit substantial identity with SEQ ID NOS: 3, 4, 5, 6, and 7. Such substantial identity refers to sequences that, when aligned to correspond as closely as possible to the sequence of the present disclosure and analyzed using standard algorithms commonly used in the field, exhibit at least 80% homology, preferably at least 85% homology, more preferably at least 90% homology, and most preferably at least 95% homology. Alignment methods for sequence comparison are well known in the art. Various alignment methods and algorithms are disclosed in Smith and Waterman, Adv. Appl. Math. 2:482 (1981); Needleman and Wunsch, J. Mol. Bio. 48:443 (1970); Pearson and Lipman, Methods in Mol. Biol. 24:307-31 (1988); Higgins and Sharp, Gene 73:237-44 (1988); Higgins and Sharp, CABIOS 5:151-3 (1989); Corpet et al., Nuc. Acids Res. 16:10881-90 (1988); Huang et al., Comp. Appl. BioSci. 8:155-65 (1992); and Pearson et al., Meth. Mol. Biol. 24:307-31 (1994). The NCBI Basic Local Alignment Search Tool (BLAST) (Altschul et al., J. Mol. Biol. 215:403-10 (1990)) is available through the National Center for Biological Information (NCBI) and can be used in conjunction with sequence analysis programs such as blastp, blastm, blastx, tblastn, and tblastx.

As polypeptides used in the present disclosure, biologically functional equivalents that are amino acid sequence variants, but exhibit the same biological activity as the polypeptide including at least one amino acid sequence selected from the group consisting of SEQ ID NOS: 3, 4, 5, 6, and 7 may also be used. Such amino acid variants are introduced based on the relative similarity of amino acid side chain substituents, for example, hydrophobicity, hydrophilicity, charge, and size. An analysis of the size, shape, and types of amino acid side chain substituents indicates that arginine, lysine, and histidine are positively charged residues; alanine, glycine, and serine have similar sizes; and phenylalanine, tryptophan, and tyrosine have similar shapes. Thus, based on the analysis, arginine, lysine, and histidine; alanine, glycine, and serine; and phenylalanine, tryptophan, and tyrosine may be considered biologically functional equivalents.

When introducing variations, the hydropathic index of amino acids may be considered. Each amino acid is assigned a hydrophobicity index based on its hydrophobicity and charge: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5). The hydropathic index of amino acid is highly significant in conferring interactive biological functions on proteins. It is a well-known fact that substituting amino acids with similar hydropathic indices preserves similar biological activity. When introducing variations with reference to the hydropathic index, substitutions are preferably made between amino acids with a hydropathic index difference of +2 or less, more preferably +1 or less, and even more preferably +0.5 or less.

Furthermore, it is well known that substitutions between amino acids with similar hydrophilicity values result in proteins with equivalent biological activity. As disclosed in U.S. Pat. No. 4,554,101, the following hydrophilicity values are assigned to each amino acid residue: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); and tryptophan (−3.4). When introducing variations with reference to hydrophilicity values, substitutions are preferably made between amino acids with a hydrophilicity value difference of +2 or less, more preferably +1 or less, and even more preferably +0.5 or less.

The exchange of amino acids in proteins that does not alter molecular activity is well known in the art (H. Neurath, R. L. Hill, The Proteins, Academic Press, New York, 1979). The most commonly occurring exchanges include substitutions between: Ala/Ser, Val/Ile, Asp/Glu, Thr/Ser, Ala/Gly, Ala/Thr, Ser/Asn, Ala/Val, Ser/Gly, Thr/Phe, Ala/Pro, Lys/Arg, Asp/Asn, Leu/Ile, Leu/Val, Ala/Glu, and Asp/Gly.

It would be apparent to a person skilled in the art that the nucleotide sequence encoding for the polypeptide of the present disclosure is sufficient as long as it encodes the amino acid sequences of peptides CBRV-1 to CBRV-5 and is not limited to any specific nucleotide sequence.

This is because even if a variation occurs in the nucleotide sequence, the expressed protein may remain unchanged in its sequence. This phenomenon is referred to as the degeneracy of the genetic code. Therefore, the nucleotide sequence may include functionally equivalent codons or codons encoding the same amino acid (e.g., due to the degeneracy of the genetic code, there are six codons for arginine or serine) or codons encoding biologically equivalent amino acids.

According to another aspect thereof, the present disclosure provides a nucleic acid molecule including a nucleotide sequence encoding for a polypeptide including at least one amino acid sequence selected from the group consisting of SEQ ID NOS: 3, 4, 5, 6, and 7.

It would be apparent to a person skilled in the art that the nucleotide sequence encoding for the polypeptide of the present disclosure is sufficient as long as it encodes the amino acid sequences of peptides CBRV-1 to CBRV-5 and is not limited to any specific nucleotide sequence.

This is because even if a variation occurs in the nucleotide sequence, the expressed protein may remain unchanged in its sequence. This phenomenon is referred to as the degeneracy of the genetic code. Therefore, the nucleotide sequence may include functionally equivalent codons or codons encoding the same amino acid (e.g., due to the degeneracy of the genetic code, there are six codons for arginine or serine) or codons encoding biologically equivalent amino acids.

In an embodiment of the present disclosure, the nucleotide sequence is SEQ ID NO: 2.

As used herein, the term “nucleic acid molecule” is intended to comprehensively include DNA (gDNA and cDNA) and RNA molecules. The nucleotides, which are the basic structural units of nucleic acid molecules, include not only naturally occurring nucleotides but also analogs with modified sugar or base moieties (Scheit, Nucleotide Analogs, John Wiley, New York (1980); Uhlman and Peyman, Chemical Reviews, 90:543-584 (1990)).

In a specific embodiment of the present disclosure, the sequence of the polypeptide including the amino acid sequence of peptide CBRV1-04369 or a part thereof is listed in the sequence listing attached to this specification.

A nucleic acid molecule comprising a nucleotide sequence encoding a polypeptide including at least one amino acid sequence selected from the group consisting of SEQ ID NOS: 3, 4, 5, 6, and 7 is interpreted to include nucleotide sequences exhibiting substantial identity with the aforementioned nucleotide sequence. The substantial identity of such sequences refers to nucleotide sequences that, when aligned to best correspond to the nucleotide sequence of the present disclosure and analyzed using standard algorithms commonly used in the field, exhibit at least 80% homology, more preferably at least 90% homology, and most preferably at least 95%, 97%, 98%, or 99% homology.

Considering the biological equivalency of the variations mentioned above, a nucleic acid molecule comprising a nucleotide sequence encoding a polypeptide including at least one amino acid sequence selected from the group consisting of SEQ ID NOS: 3, 4, 5, 6, and 7 is interpreted to include sequences that exhibit substantial identity with the sequence listed in the sequence listing. The substantial identity in this case refers to sequences that, when aligned as closely as possible to the sequence of the present disclosure and analyzed using commonly used algorithms in the field, exhibit at least 61% homology, more preferably 70% homology, even more preferably 80% homology, and most preferably 90% homology. Methods for sequence alignment for sequence comparison are well known in the field. Various alignment methods and algorithms are disclosed in: Smith and Waterman, Adv. Appl. Math. 2:482 (1981); Needleman and Wunsch, J. Mol. Bio. 48:443 (1970); Pearson and Lipman, Methods in Mol. Biol. 24:307-31 (1988); Higgins and Sharp, Gene 73:237-44 (1988); Higgins and Sharp, CABIOS 5:151-3 (1989); Corpet et al., Nuc. Acids Res. 16:10881-90 (1988); Huang et al., Comp. Appl. BioSci. 8:155-65 (1992); Pearson et al., Meth. Mol. Biol. 24:307-31 (1994). The NCBI Basic Local Alignment Search Tool (BLAST) (Altschul et al., J. Mol. Biol. 215:403-10 (1990)) is accessible through the National Center for Biological Information (NCBI). It can be used with sequence analysis programs such as blastp, blastn, blastx, tblastn, and tblastx. BLAST is accessible via the BLAST page on the NCBI website, and the method for sequence homology comparison using this program can be found on the BLAST help page of the NCBI website.

According to another aspect thereof, the present disclosure provides a recombinant vector comprising a nucleic acid molecule including a nucleotide sequence encoding for a polypeptide including at least one amino acid sequence selected from the group consisting of SEQ ID NOS: 3, 4, 5, 6, and 7.

As used herein, the term “vector” refers to a means for expressing a target gene in a host cell and encompasses plasmid vectors, cosmid vectors, bacteriophage vectors, and viral vectors such as adenovirus vectors, retrovirus vectors, and adeno-associated virus vectors.

According to a specific embodiment of the present disclosure, the nucleic acid molecule including a nucleotide sequence encoding for a polypeptide including at least one amino acid sequence of SEQ ID NOS: 3, 4, 5, 6, and 7 is operatively linked to a promoter in the vector of the present disclosure.

The term “operatively linked,” as used herein, refers to a functional connection between a nucleic acid expression regulatory sequence (e.g., promoter, signal sequence, or an array of transcription factor binding sites) and another nucleic acid sequence, whereby the regulatory sequence controls the transcription and/or translation of the other nucleic acid sequence.

The recombinant vector system of the present disclosure can be constructed using various methods known in the art, and specific methods for constructing such a system are disclosed in Sambrook et al., Molecular Cloning, A Laboratory Manual, Cold Spring Harbor Laboratory Press (2001), which is incorporated herein by reference.

The vector of the present disclosure can be designed as a vector for gene cloning or as an expression vector for protein expression. Furthermore, the vector of the present disclosure can be constructed using either a prokaryotic or eukaryotic host cell.

For example, when the vector of the present disclosure is an expression vector and uses a eukaryotic cell as a host, promoters derived from the genome of mammalian cells (e.g., metallothionein promoter, beta-actin promoter, human hemoglobin promoter, and human muscle creatine promoter) or promoters derived from mammalian viruses (e.g., adenovirus late promoter, vaccinia virus 7.5K promoter, SV40 promoter, cytomegalovirus (CMV) promoter, HSV tk promoter, mouse mammary tumor virus (MMTV) promoter, HIV LTR promoter, Moloney virus promoter, Epstein-Barr virus (EBV) promoter, and Rous sarcoma virus (RSV) promoter) may be used.

These promoters are generally accompanied by a transcription termination sequence, such as a polyadenylation sequence.

The vector of the present disclosure may be fused with other sequences to facilitate the purification of the expressed polypeptide or protein. The fused sequences may include, for example: glutathione S-transferase (Pharmacia, USA), maltose-binding protein (NEB, USA), FLAG tag (IBI, USA), and 6× His (hexahistidine) tag (Qiagen, USA).

The expression vector of the present disclosure includes an antibiotic resistance gene as a selection marker, which is commonly used in the field. Examples include resistance genes for: ampicillin, gentamicin, carbenicillin, chloramphenicol, streptomycin, kanamycin, geneticin, neomycin, and tetracycline.

According to another aspect thereof, the present disclosure provides a host cell transformed with the recombinant vector.

Host cells capable of stably and continuously cloning and expressing the vector of the present disclosure are well known in the art, and any host cell may be used. For example, suitable eukaryotic host cells for the vector include, but are not limited to, COS7 (monkey kidney cells), NSO cells, SP2/0 cells, Chinese hamster ovary (CHO) cells, BHK (baby hamster kidney) cells, MDCK cells, myeloma cell lines, HuT 78 cells, and HEK-293 cells.

As used herein, the terms “transformed,” “transduced,” or “transfected” refer to the process in which an exogenous nucleic acid is delivered or introduced into a host cell. A “transformed,” “transduced,” or “transfected” cell is a cell that has been genetically modified with an exogenous nucleic acid, and the term includes both the transformed cell itself and its progeny derived from serial passages.

According to another aspect thereof, the present disclosure provides a pharmaceutical composition for preventing or treating amyloid beta-related neurodegenerative diseases, comprising a polypeptide including at least one amino acid sequence selected from the group consisting of SEQ ID NOS: 3, 4, 5, 6, and 7.

In an embodiment of the present disclosure, the polypeptide inhibits amyloid beta fibrillation and oligomerization.

As used herein, the term “amyloid beta” refers to a group of proteins consisting of 39 to 43 amino acids, generated by sequential hydrolysis of amyloid precursor protein (APP) by beta- and gamma-secretases (β-, γ-secretases).

The term “amyloid beta 42” (Aβ42), as used herein, refers to an aggregation-prone amyloid beta protein with an Ala residue at the C-terminal, which is known to be predominantly accumulated in the brains of Alzheimer's disease patients from the early stages.

As used herein, the term “amyloid beta oligomer” refers to a soluble or insoluble form in which amyloid beta monomers are aggregated.

As used herein, the term “amyloid beta oligomerization” refers to the process by which amyloid beta monomers aggregate into soluble or insoluble oligomers.

The term “neurodegenerative disease,” as used herein, refers to a disease selected from the group consisting of Alzheimer's disease, Parkinson's disease, Huntington's disease, mild cognitive impairment, amyloid angiopathy, amyloid-related stroke, and systemic amyloidosis.

As used herein, the term “Alzheimer's disease” (AD) refers to a progressive neurodegenerative disease characterized by the formation of amyloid plaques due to amyloid beta deposition in brain regions such as the cerebral cortex and hippocampus, and by neurofibrillary tangles formed when tau protein binds to microtubules. Alzheimer's disease is accompanied by gradual memory loss, cognitive decline, and dementia. Clinical symptoms of Alzheimer's disease include memory impairment, language deterioration, reduced spatial awareness, decline in judgment and ability to perform daily activities, gait disorders, and motor dysfunction.

As used herein, the term “prevention” refers to the prevention or protective treatment of a disease or disease state, while the term “treatment” refers to the reduction, suppression, alleviation, or elimination of a disease state.

The pharmaceutical composition of the present disclosure may be formulated into powders, granules, tablets, coated tablets, pills, sugar-coated tablets, capsules, solutions, suspensions, gels, syrups, slurries, suppositories, enemas, emulsions, pastes, ointments, creams, lotions, sprays, or suspensions.

The pharmaceutical composition of the present disclosure may further include appropriate carriers, excipients, or diluents that are commonly used in pharmaceutical formulations. Examples include lactose, dextrose, sucrose, sorbitol, mannitol, xylitol, erythritol, starch, acacia gum, alginates, gelatin, calcium phosphate, calcium silicate, cellulose, methylcellulose, microcrystalline cellulose, polyvinylpyrrolidone, water, methylparaben, propylparaben, talc, magnesium stearate, and mineral oils.

The pharmaceutical composition of the present disclosure is administered in a pharmaceutically effective amount. The “pharmaceutically effective amount” refers to an amount sufficient to treat a disease with a reasonable benefit/risk ratio applicable to medical treatment. The effective dose level may vary depending on factors such as the type and severity of the disease, drug activity, patient sensitivity to the drug, administration time, route of administration, excretion rate, treatment period, concomitant medications, and other factors well known in the medical field.

The dosage of the composition may vary depending on the age, gender, and body weight of the patient, but it can be administered once or multiple times daily in an amount sufficient to maintain a therapeutically effective blood concentration for treating amyloid-related diseases.

The dosage of the composition may be increased or decreased depending on the route of administration, severity of the disease, gender, body weight, and age. Therefore, the dosage is not intended to limit the scope of the present disclosure in any way.

The pharmaceutical composition of the present disclosure may be administered via various routes to a subject. All possible routes of administration are contemplated, including: intracerebral administration, oral administration, subcutaneous injection, intraperitoneal injection, intravenous injection, intramuscular injection, intrathecal (spinal) injection, sublingual administration, buccal (oral mucosal) administration, rectal administration, vaginal administration, ocular administration, auricular (ear) administration, intranasal administration, inhalation, oral or nasal spray, topical (dermal) administration, and transdermal administration.

The pharmaceutical composition of the present disclosure may be administered as an individual therapeutic agent or in combination with other therapeutic agents. It can be administered sequentially or simultaneously with conventional therapeutic agents and can be administered as single or multiple doses. Considering all these factors, it is important to administer the minimum amount necessary to achieve the maximum effect without side effects, which can be easily determined by a person skilled in the art.

In the present disclosure, “prevention” refers to any action that inhibits or delays the onset of Alzheimer's disease, while “treatment” refers to any action that improves or beneficially modifies Alzheimer's disease due to the administration of the pharmaceutical composition of the present disclosure. “Improvement” refers to any action that reduces disease-related parameters, such as symptom severity, due to administration of the composition of the present disclosure.

According to another aspect thereof, the present disclosure provides a composition for detecting amyloid beta protein, comprising a polypeptide including at least one amino acid sequence selected from the group consisting of SEQ ID NOS: 3, 4, 5, 6, and 7.

The composition for detecting amyloid beta protein of the present disclosure may further include additives commonly contained in residual drug detection compositions, such as auxiliary substances for maintaining the activity of the peptide and for confirming the binding between the peptide and amyloid beta protein.

The composition for detecting amyloid beta protein of the present disclosure may further include tools, devices, or reagents commonly used in diagnostic compositions that employ peptides to determine the presence or absence of amyloid beta protein in biological samples. These may include tools for collecting test samples or applying diagnostic reagents, and reagents or devices for confirming the binding between at least one amino acid sequence selected from the group consisting of SEQ ID NOS: 3, 4, 5, 6, and 7 and amyloid beta protein.

The tools and reagents that may be included in the detection composition include, but are not limited to, a suitable carrier, labeling substances capable of generating detectable signals, solvents, detergents, buffers, and stabilizers. A suitable carrier may include, but is not limited to, soluble carriers, such as physiologically acceptable buffer solutions known in the art (e.g., PBS), insoluble carriers, such as polystyrene, polyethylene, polypropylene, polyester, polyacrylonitrile, fluororesins, crosslinked dextran, polysaccharides, and polymeric materials such as magnetic microparticles coated with metals, as well as paper, glass, metal, agarose, or combinations thereof.

As used herein, the term “multimer detection system” (MDS) refers to a method for differentially detecting multimeric forms from monomeric forms of a multimer-forming polypeptide. More specifically, it may be the detection method disclosed in Korean Patent No. 10-0987639.

As used herein, the term “thioflavin T (ThT) assay” refers to an analytical technique used to quantify the extent of fibril formation and inhibition of amyloid proteins in the presence of anti-amyloidogenic compounds.

According to another aspect thereof, the present disclosure provides a method for detecting amyloid beta protein, comprising the step of detecting a peptide-amyloid beta protein complex formed by contacting the amyloid beta protein detection composition with a sample.

According to an embodiment of the present disclosure, the detection step may be performed using any experimental method, such as ELISA or surface plasmon resonance (SPR) techniques, capable of confirming binding signals between proteins and peptides.

### Advantageous Effects of Invention

The features and advantages of the present disclosure are summarized as follows:
(a) The present disclosure provides a polypeptide including at least one amino acid sequence selected from the group consisting of SEQ ID NOS: 3, 4, 5, 6, and 7.
(b) The present disclosure provides a nucleic acid molecule including a nucleotide sequence encoding for a polypeptide that includes at least one amino acid sequence selected from the group consisting of SEQ ID NOS: 3, 4, 5, 6, and 7.
(c) The present disclosure provides a recombinant vector comprising a nucleic acid molecule including a nucleotide sequence encoding for a polypeptide that includes at least one amino acid sequence selected from the group consisting of SEQ ID NOS: 3, 4, 5, 6, and 7.
(d) The present disclosure provides a host cell transformed with a recombinant vector comprising a nucleic acid molecule including a nucleotide sequence encoding for a polypeptide that includes at least one amino acid sequence selected from the group consisting of SEQ ID NOS: 3, 4, 5, 6, and 7.
(e) The present disclosure provides a pharmaceutical composition for the prevention or treatment of amyloid beta-related neurodegenerative diseases, comprising a polypeptide including at least one amino acid sequence selected from the group consisting of SEQ ID NOS: 3, 4, 5, 6, and 7.
(f) The present disclosure provides a composition for detecting amyloid beta protein, comprising a polypeptide including at least one amino acid sequence selected from SEQ ID NOS: 3, 4, 5, 6, and 7.
(g) The present disclosure provides a method for detecting amyloid beta protein, comprising a step of detecting a peptide-amyloid beta protein complex formed by contacting a sample with a composition for detecting amyloid beta protein that contains a polypeptide including at least one amino acid sequence selected from SEQ ID NOS: 3, 4, 5, 6, and 7.
(h) When used, a polypeptide including at least one amino acid sequence selected from SEQ ID NOS: 3, 4, 5, 6, and 7 of the present disclosure, which directly binds to amyloid beta protein, can prevent amyloid beta fibrillation and oligomerization, thus finding applications in the prevention and treatment of amyloid-related diseases such as Alzheimer's disease. Additionally, with the ability to detect amyloid beta, the peptide can also be utilized for the diagnosis of amyloid-related diseases, including Alzheimer's disease.

### BRIEF DESCRIPTION OF THE DRAWINGS

FIG. 1 illustrates the structure and amino acid sequence of peptide CBRV1-04369. The peptide CBRV1-04369 in FIG. 1 contains six cysteine residues with three disulfide bonds formed therein. These disulfide bonds are formed between the first and sixth cysteine residues, the second and fourth cysteine residues, and the third and fifth cysteine residues. Specifically, in FIG. 1, Cys at position 1 forms a disulfide bond with Cys at position 36, Cys at position 9 forms a disulfide bond with Cys at position 29, and Cys at position 18 forms a disulfide bond with Cys at position 33.

FIG. 2 shows the inhibitory effect on amyloid beta protein (Aβ) fibrillation, as measured using the Thioflavin T (ThT) assay. In the CBRV1-04369 peptide-treated group, a significant inhibition of Aβ fibrillation was observed compared to the negative control group, which received no additional substances, and the positive control group, which was treated with doxorubicin, a known inhibitor of amyloid beta aggregation.

FIG. 3 presents the inhibitory effect of the peptide CBRV1-04369 on amyloid beta protein (Aβ) oligomerization, as assessed by the multimer detection system (MDS) assay. The results indicate that peptide CBRV1-04369 effectively inhibits Aβ oligomerization.

FIG. 4 demonstrates the direct binding affinity of peptide CBRV1-04369 to amyloid beta42 (Aβ42). The peptide was immobilized on a hydrophilic plate surface, followed by Aβ42 treatment to induce binding, and the binding interaction was quantitated using ELISA with anti-Aβ42 antibodies. Both standard ELISA and maleimide ELISA results showed high signal intensities, confirming strong binding of peptide CBRV1-04369 to Aβ42.

FIG. 5 illustrates whether treatment with peptide CBRV1-04369 mitigates Aβ-induced cytotoxicity. When SH-SY5Y neuroblastoma cells were treated with peptide CBRV1-04369 and co-cultured with Aβ1-42 (5 M), an increase in cell viability was observed.

FIG. 6 illustrates the results of identifying the key sequence within the CBRV1-04369 peptide that exhibits inhibitory effects on Aβ fibrillization and oligomerization.

FIG. 7 illustrates the experimental results verifying the effect of peptide treatment on the recovery of spatial memory.

FIGS. 8 and 9 illustrate the experimental results verifying the effect of peptide treatment on the recovery of object memory.

FIG. 10 illustrates the experimental results verifying the effect of peptide treatment on the recovery of memory related to stimuli.

FIG. 11 illustrates the experimental results verifying the inhibitory effect of CBRV-1 peptide treatment on amyloid plaque formation.

FIG. 12 illustrates the experimental results verifying the inhibitory effect of CBRV-4 peptide treatment on amyloid plaque formation.

FIG. 13 illustrates the analysis results of the inhibitory effect of CBRV-1 peptide treatment on Aβ oligomerization by measuring Aβ oligomers of a specific molecular weight using Western blot analysis of mouse brain homogenates with the A11 antibody. (A) shows the results of Aβ oligomer analysis in the contralateral and ipsilateral sides, respectively. (B) shows the results of Aβ oligomer analysis combining the contralateral and ipsilateral sides. (C) shows the Western blot results of Aβ oligomers.

FIG. 14 illustrates the analysis results of the inhibitory effect of CBRV-4 peptide treatment on Aβ oligomerization by measuring Aβ oligomers of a specific molecular weight using Western blot analysis of mouse brain homogenates with the 6E10 antibody. (A) shows the results of Aβ oligomer analysis in the contralateral and ipsilateral sides, respectively. (B) shows the results of Aβ oligomer analysis combining the contralateral and ipsilateral sides. (C) shows the Western blot results of Aβ oligomers.

FIG. 15 illustrates the verification of changes in inflammation-related marker levels upon peptide treatment.

### MODE FOR CARRYING OUT THE INVENTION

A better understanding of the present disclosure may be obtained through the following examples which are set forth to illustrate the present disclosure in more detail, and it will be apparent to those skilled in the art that the scope of the invention is not limited to the examples.

### EXAMPLES

Throughout this specification, the “%” used to indicate the concentration of a specific substance refers to: (w/w) % for solid/solid, (w/v) % for solid/liquid, and (v/v) % for liquid/liquid, unless otherwise specified.

### Example 1: Experimental Methods

### 1-1. Synthesis of Peptide CBRV1-04369

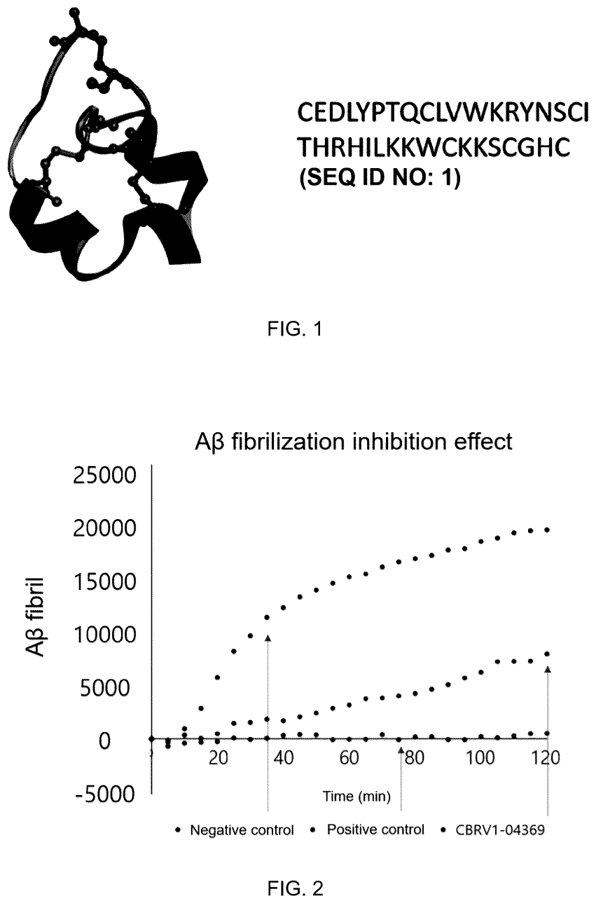

Synthesis of peptide CBRV1-04369, as shown in FIG. 1, was commissioned to and made by Shanghai Royobiotech Co., Ltd. The peptide used in the present disclosure is preferably synthesized using conventional chemical peptide synthesis methods (W. H. Freeman and Co, Proteins; Structures and molecular principles, 1983). Specifically, the synthesis may be conducted by solution-phase peptide synthesis, solid-phase peptide synthesis, fragment condensation methods, or using F-moc or T-BOC chemistry. Among these, solid-phase peptide synthesis is the most preferred method.

### 1-2. ThT Assay

Human Aβ42 (AggreSure™; AnaSpec) was dissolved in PBS to prepare amyloid beta protein. The peptide was added to the amyloid beta solution, followed by the addition of Thioflavin T (ThT) solution (50 μM; Sigma Aldrich). The mixture was incubated at 37 °C for 24 hours. After incubation, the fluorescence signal was measured using a microplate reader (PerkinElmer Victor-3®). The negative control was prepared by mixing ThT solution and amyloid beta protein without any additional peptide or compound.

### 1-3. MDS Assay

First, amyloid beta capturing antibody 6E10 (BioLegend, USA) was diluted in a sodium carbonate-bicarbonate coating buffer (pH 9.4). The coating buffer thus prepared was dispensed into a 96-well plate and incubated overnight at 4 °C to coat the plate with the capturing antibody.

The coated plate was then blocked by dispensing blocking buffer and incubating at room temperature for 2 hours. The blocking buffer was PBST containing 2% bovine serum albumin (BSA). After blocking, the sample solutions were added to the wells and incubated at room temperature for 1 hour.

Next, the detecting buffer was dispensed to each well and incubated at room temperature for 30 minutes. The detecting buffer was PBST containing WO2-horseradish peroxidase (WO2-HRP) antibody (0.1 μg/ml; PeopleBio Inc., South Korea).

Then, TMB solution (3,3′,5,5′-tetramethylbenzidine) was added and incubated for 30 minutes at room temperature. After incubation, stop solution (1 M H₂SO₄) was added to each well. The signal was read at 450 nm using a microplate reader (PerkinElmer Victor-3®). The negative control was also measured at 450 nm.

### 1-4. ELISA

For standard ELISA, the polypeptide was diluted in sodium carbonate-bicarbonate buffer (pH 9.4) and added to a microplate, followed by overnight incubation at 4 °C. The coated plate was blocked by adding blocking buffer and incubating at room temperature for 2 hours. The blocking buffer was PBST containing 2% bovine serum albumin (BSA). After blocking, Aβ42 was added to allow binding to the polypeptide. Subsequently, the detecting buffer was added to each well and incubated at room temperature for 30 minutes. The detecting buffer was PBST containing the detecting antibody WO2-horseradish peroxidase (WO2-HRP) antibody (0.1 μg/ml; PeopleBio Inc., South Korea). TMB solution (3,3′,5,5′-tetramethylbenzidine) was then added and incubated for 30 minutes. Stop solution (H₂SO₄, 1 M) was added to each well. The signal was read at 450 nm using a microplate reader (PerkinElmer Victor-3®). The negative control was also measured at 450 nm.

For maleimide ELISA, maleimide-activated plates (Thermo) were treated with polypeptide diluted in PBS and incubated overnight at 4 °C. The coated plate was blocked using blocking buffer at room temperature for 2 hours. The blocking buffer was PBST containing 2% bovine serum albumin (BSA). After blocking, Aβ42 was added to and reacted with the polypeptide. Subsequently, a detecting buffer was added to each well, followed by incubation at room temperature for 30 minutes. The detecting buffer was PBST containing 0.1 μg/ml of the detecting antibody WO2-HRP (WO2-horseradish peroxidase antibody, PeopleBio Inc., South Korea) antibody. TMB solution (3,3′,5,5′-tetramethylbenzidine) was added to each well and incubated for 30 minutes. Stop solution (1 M H₂SO₄) was added to each well. Then, the signal was read at 450 nm using a microplate reader (PerkinElmer Victor-3®). The negative control was also measured at 450 nm.

### 1-5. ATP Luminescence Assay

Cells were seeded at a density of 2×10 cells/well in DMEM medium into a 96-well plate and stabilized overnight. The control group was treated with amyloid beta only while the experimental groups were treated with polypeptide and amyloid beta together for 24 hours. After incubation, the culture medium was removed, and 0.5 mg/ml MTT solution (100 μl/well) was added. The plate was incubated at 37 °C for at least 1 hour in an incubator. After removing the MTT solution, 200 μl of DMSO was added. The absorbance of the formazan thus formed was read at 540 nm on an ELISA reader.

### 1-6. Animal Surgery and Drug Administration Method

For drug administration, an Alzet osmotic pump, an implantable drug delivery device based on osmotic pressure for animal experiments, was used. The osmotic pump (Model 2006, Alzet DURECT Corporation, Cupertino, CA; pumping rate=0.15 μl/hr, duration=42 days, reservoir volume=200 μl) is a small, capsule-shaped, implantable pump that continuously releases 0.15 μl of the drug per hour for a total of 42 days, approximately six weeks.

Experimental animals were anesthetized with an intraperitoneal injection of a mixture of Zoletil 50 and Rompun at a volume ratio of 1:2, at doses of 16.7 mg/kg for Zoletil and 15.5 mg/kg for Rompun, before surgery.

Using a stereotaxic system, the animal's head was fixed, and the osmotic pump was implanted subcutaneously in the upper back. A syringe (cannula) connected to the osmotic pump was inserted 0.5 mm posterior and 1 mm lateral to the bregma, followed by suturing of the surgical site.

The experimental groups received 200 μl of either CBRV1-04369 or CBRV-4 at a concentration of 25 mg/ml into the reservoir, while the control group received 200 μl of saline into the reservoir.

### 1-7. Animal Behavioral Experiments

Behavioral experiments were conducted on an Alzheimer's disease animal model after six weeks of drug administration to evaluate memory recovery. The behavioral experiments included the Y-maze test, passive avoidance test, and novel object recognition (NOR) test. Before all behavioral experiments, food was withheld for one hour.

### Y-Maze Test

In the Y-maze test, mice were placed in a Y-shaped maze and allowed to freely explore for eight minutes. Each time a mouse entered an arm of the maze, the sequence of entries was recorded. Total alternation was calculated as the number of times the mouse sequentially entered different arms in succession, regardless of order. Total arm entries were also measured to assess locomotor activity.

Spontaneous alterations (%)=(Total number of alternations)/(Total number of arm entries−2)

### Novel Object Recognition (NOR) Test

This test, conducted over three days, was designed to assess attention span. On the first day, mice were placed in a box for five minutes to acclimate to the environment. On the second day, two objects were placed inside the box, and the time spent interacting with each object was recorded for ten minutes. On the third day, one object was replaced with a novel object, and the time spent with the novel object was recorded for ten minutes.

### Passive Avoidance Test

The passive avoidance test was conducted using the Gemini avoidance system over three days. On the first day, mice were trained to move from a bright chamber to a dark chamber within the Gemini system. Each mouse was placed in the bright chamber, and its behavior was observed for five minutes. The training continued until the mouse entered the dark chamber within 20 seconds. If the mouse did not enter the dark chamber within five minutes, the experimenter guided it into the dark chamber to complete the training.

On the second day, an electric shock of 0.2 mA was administered for five seconds when the mouse entered the dark chamber. The mouse's vocalization was used to confirm the reception of the stimulus.

On the final day, the mouse was placed in the bright chamber, and the latency time (the time taken to enter the dark chamber) was measured over five minutes.

### 1-8. Blood Collection and Brain Tissue Sampling

After behavioral experiments, experimental animals from each group were anesthetized with an intraperitoneal injection of Zoletil 50 and Rompun at a volume ratio of 1:2 (Zoletil 16.7 mg/kg, Rompun 15.5 mg/kg). Blood was collected for MDS analysis, and serum was separated.

The brain tissue was then extracted via perfusion. The anterior part of the brain tissue, relative to the osmotic pump implantation site, was used for Western blot analysis, while the posterior part was used for Thioflavin S staining.

The posterior brain tissue was fixed in 4% PFA and subsequently dehydrated in 30% sucrose overnight. The tissue was then subjected to frozen sectioning, yielding 30 μm-thick brain tissue sections.

### 1-9. Thioflavin S Staining

Thioflavin S staining was performed to evaluate amyloid plaque deposition in the hippocampus and cortex regions of the brain. Brain tissue sections were stained in a 0.1% Thioflavin-S solution dissolved in 70% ethanol for 10 minutes, followed by washing with PBS-T and mounting. Fluorescence microscopy was used to capture images of amyloid plaque deposition in the hippocampus and cortex. The number of plaques and their intensity were quantified.

### 1-10. Western Blot Analysis

Western blot analysis was performed on brain homogenates. Frozen brain tissues were homogenized using beads, and the resulting brain homogenates were stored at −80 °C. The total protein concentration was measured using the BCA assay and diluted to a consistent concentration in PBS.

For Western blot analysis, uniformly diluted brain homogenates were used. The homogenates were transferred onto membranes and probed with 6E10 and A11 antibodies to detect Aβ in various molecular weight forms. To ensure clear detection of oligomers, the upper and lower parts of the membrane were separated before analysis.

### Example 2: Selection and Validation of Candidate Substances for Alzheimer's Disease Treatment

Candidate substances capable of inhibiting oligomerization and fibrillization of amyloid beta (Aβ) protein, which is the causative agent of Alzheimer's disease, were screened. Conventionally, substances that inhibit Aβ protein fibrillization have been selected as candidate compounds for Alzheimer's disease treatment. However, given that: the oligomeric form of amyloid beta protein is suggested to have cytotoxicity; and inhibition of fibrillization may increase the stability of oligomers, potentially leading to adverse effects, it is possible that screening based solely on fibrillization inhibition may not be an appropriate method for selecting effective therapeutic compounds. Therefore, in the present disclosure, CBRV1-04369 was selected as a candidate Alzheimer's disease therapeutic agent based on its ability to inhibit both Aβ protein oligomerization and fibrillization.

### 2-1. Verification of Aβ Fibrillization Inhibition by CBRV1-04369 Via ThT Assay

The inhibition of CBRV1-04369 against Aβ fibrillization was evaluated through thioflavin T (ThT) assay. In this regard, the peptide CBRV1-04369, Thioflavin T, and Aβ were reacted together, and fluorescence intensity was monitored to assess the inhibitory effect on Aβ fibrillization. Fluorescence measurements were taken every 5 minutes for a total of 2 hours.

The results are shown in FIG. 2.

As shown in FIG. 2, compared to the negative control, Aβ fibrillization was inhibited by approximately 86.4% in the CBRV1-04369-treated group, confirming that CBRV1-04369 exhibits a strong inhibitory effect on Aβ fibrillization.

### 2-2. Verification of Aβ Oligomerization Inhibition by CBRV1-04369 Via MDS Assay

The Multimer Detection System (MDS) assay was conducted to evaluate the inhibitory effect of CBRV1-04369 on Aβ oligomerization. Aβ was reacted with CBRV1-04369, and the resulting samples were stored at −80 °C. The level of Aβ oligomerization in each sample was then measured using the MDS-HTS system.

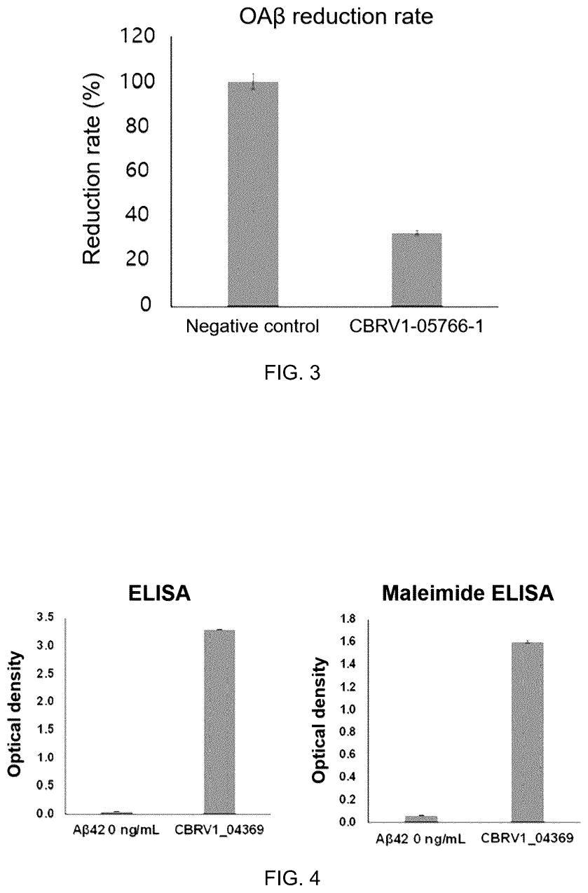

The results are shown in FIG. 3.

As shown in FIG. 3, compared to the negative control, Aβ oligomerization was inhibited by approximately 70% in the CBRV1-04369-treated group, confirming that CBRV1-04369 exhibits a strong inhibitory effect on Aβ oligomerization.

### Example 3: Direct Binding Measurement of Aβ and Peptide CBRV1-04369 Via ELISA

Plates were treated with CBRV1-04369 and Aβ through ELISA, followed by reading absorbance. For general ELISA, the peptide was immobilized on the plate surface via hydrophilic binding, followed by the addition of Aβ42 to induce binding. The presence and extent of binding were determined using an anti-Aβ42 antibody. For maleimide ELISA, the plate surface contained maleimide residues, which selectively bind to cysteine residues in the peptide. The peptide was immobilized on the plate, followed by the addition of Aβ42 to induce binding. The binding interaction was then quantified using an antibody-based detection system.

The results are shown in FIG. 4.

As shown in FIG. 4, the CBRV1-04369 peptide demonstrated strong Aβ42 binding affinity in both General ELISA and Maleimide ELISA.

### Example 4: Verification of Inhibition of Aβ-Induced Cytotoxicity

SH-SY5Y neuroblastoma cells were seeded at a density of 5,000 cells per well in 96-well plates. The cell viability was then assessed by measuring absorbance after treatment with Aβ and CBRV1-04369. An untreated control group was neither added with Aβ nor test compound. For an Aβ-treated control group, Aβ was added without any additional compounds. As for an experimental group, cells were pretreated for 5 hours with 15 UM of CBRV1-04369 peptide, followed by exposure to 5 M of Aβ1-42, and then incubated.

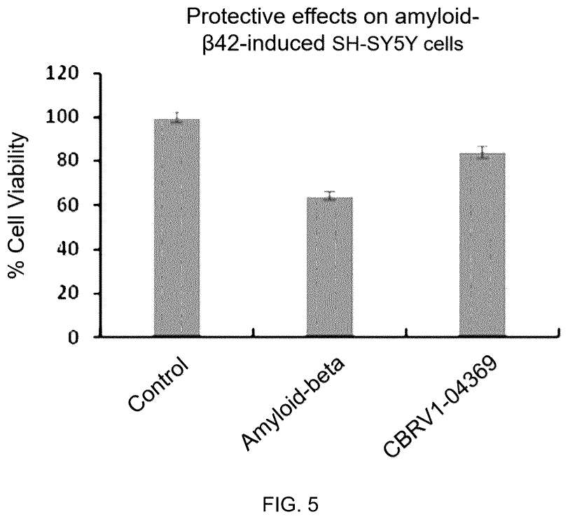

The results are shown in FIG. 5.

As shown in FIG. 5, the Aβ-treated group exhibited a cell viability of only 64%, whereas the group treated with CBRV1-04369 showed an increase in viability to 84%. This confirms that CBRV1-04369 effectively mitigates Aβ-induced cytotoxicity.

### Example 5: Identification of Key Sequences in CBRV1-04369 Peptide

To identify the functional sequences within the CBRV1-04369 peptide that exhibit inhibitory effects on Aβ fibrillization and oligomerization, ThT and MDS assays were conducted. Considering the amino acid sequence of the precursor protein of CBRV1-04369, the CNFCEEK amino acid sequence was added upstream and the RIAAPPV amino acid sequence was added downstream (SEQ ID NO: 8). The sequence was then divided into five segments, and each segment's ability to inhibit Aβ fibrillization and oligomerization was evaluated. To ensure precise assessment of each segment, amino acid C, which is highly prone to oxidation, was partially substituted with A to generate modified peptide fragments.

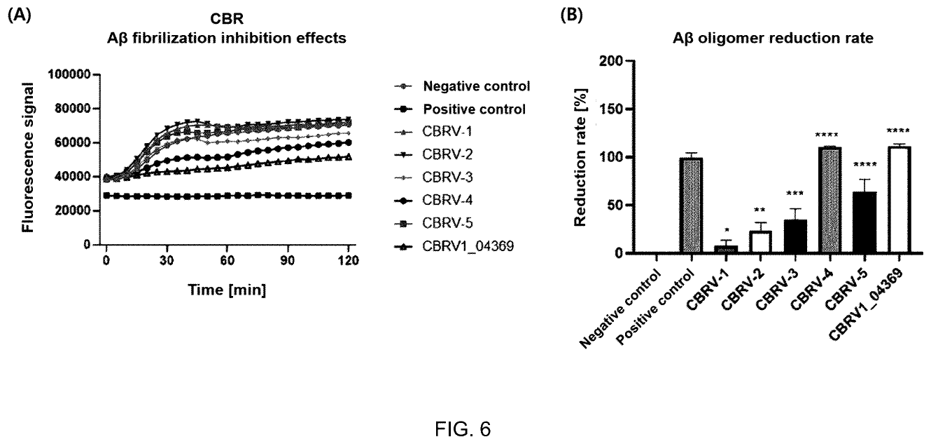

The results are shown in FIG. 6.

As illustrated in FIG. 6, CBRV-4 exhibited the strongest inhibitory effect on Aβ fibrillization and oligomerization, showing a level of effectiveness comparable to the full-length CBRV1-04369 peptide.

### Example 6: Animal Behavioral Experiments

### 6-1. Y-Maze Test

The Y-maze test was performed to evaluate spontaneous behavioral changes and spatial memory recovery.

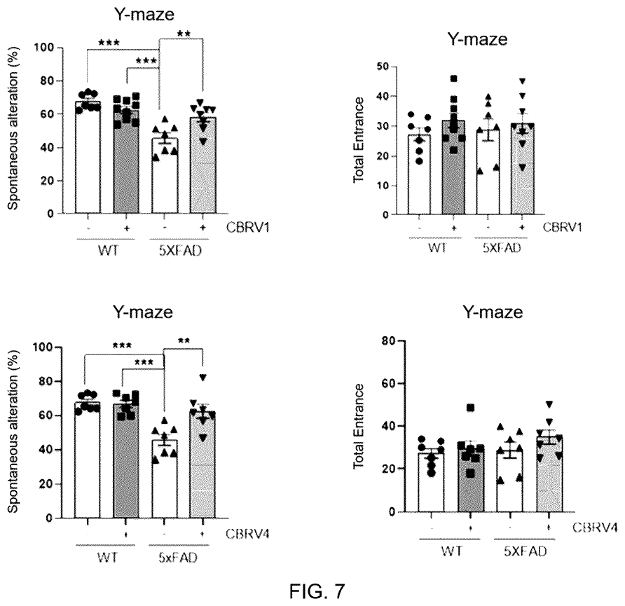

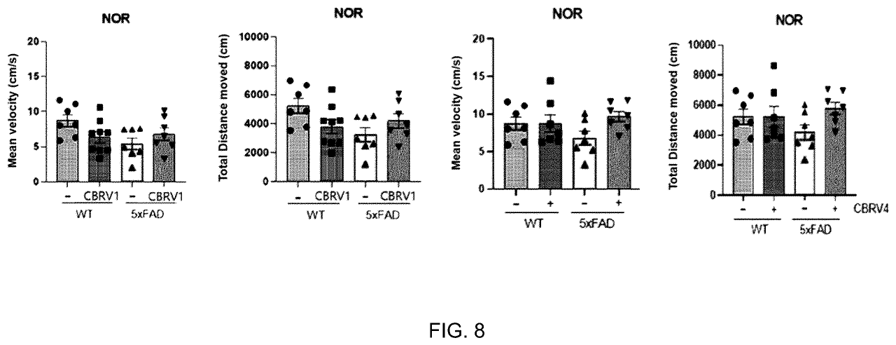

The results are shown in FIG. 7.

As depicted in FIG. 7, the number of arm entries in the Y-maze was approximately 30 to 40 times on average for each mouse group, including both wild-type (WT) and Alzheimer's disease model mice (5×FAD). The number of entries (entrances) did not significantly differ among groups. However, the 5×FAD-Vehicle group exhibited a significantly lower spontaneous alternation rate compared to the WT-Vehicle, WT-CBRV-1, and WT-CBRV-4 groups. On the other hand, the 5×FAD-CBRV-1 and 5×FAD-CBRV-4 treatment groups showed a significant increase in spontaneous alternation rate compared to the 5×FAD-Vehicle group.

These findings indicate that CBRV-1 and CBRV-4 administration may facilitate spatial memory recovery in Alzheimer's disease patients.

### 6-2. Novel Object Recognition (NOR) Test

The NOR test was conducted to assess memory recovery by measuring the time spent exploring a novel object compared to a familiar object.

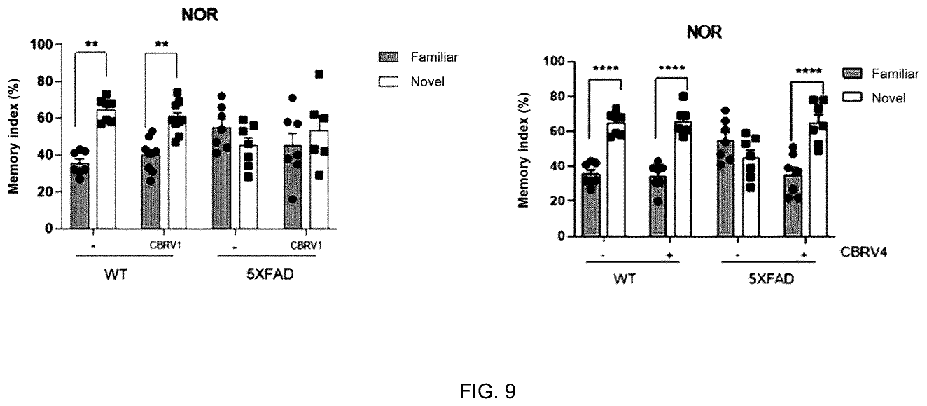

The results are shown in FIGS. 8 and 9.

As illustrated in FIG. 8, the total distance traveled and movement speed over a 10-minute period were measured for each group. In the WT groups, peptide administration did not significantly affect total movement distance or speed. However, in the Alzheimer's disease model (5×FAD), CBRV-1 and CBRV-4 administration led to a slight increase in total movement distance and speed.

As shown in FIG. 9, memory index (%) was calculated based on the time spent exploring the familiar object versus the novel object. WT-Vehicle, WT-CBRV-1, WT-CBRV-4, and 5×FAD-CBRV-4 groups exhibited a significantly higher preference for exploring the novel object compared to the familiar one. In contrast, the 5×FAD-Vehicle group spent more time exploring the familiar object than the novel one. The 5×FAD-CBRV-4 treatment group demonstrated a significantly stronger preference for the novel object compared to the 5×FAD-Vehicle group, indicating an improvement in object recognition memory.

These findings suggest that CBRV-1 and CBRV-4 administration may promote object memory recovery in Alzheimer's disease patients.

### 6-3. Passive Avoidance Test

To evaluate memory retention of an unpleasant external stimulus (electric shock), a passive avoidance test was conducted. During the training phase, mice were placed in the bright chamber of the passive avoidance box, and their natural preference for dark environments was utilized to train them to move into the dark chamber within 20 seconds. After training, mice received an electric shock upon entering the dark chamber, reinforcing the association between the dark chamber and the unpleasant stimulus. On the following day, memory retention was assessed by measuring the memory of corresponding stimuli. Mice with stronger memory retention of the stimulus exhibited a longer step-through latency time, which represents the time taken to enter the dark chamber, whereas those with impaired memory exhibited reduced step-through latency.

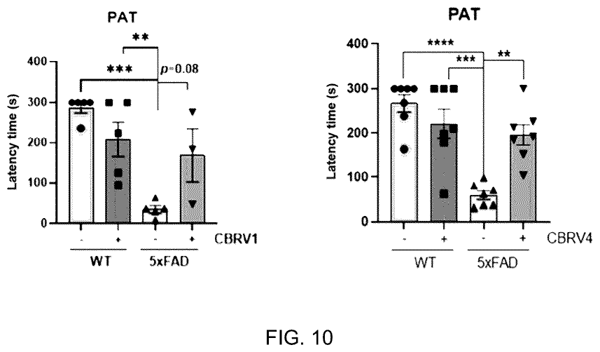

The results are shown in FIG. 10.

As depicted in FIG. 10, compared to the WT-Vehicle group, the 5×FAD-Vehicle group exhibited a significantly reduced step-through latency time, indicating impaired memory retention. However, the 5×FAD-CBRV-4 treatment group showed a significant increase in step-through latency time compared to the 5×FAD-Vehicle group, demonstrating improved memory retention.

These findings suggest that CBRV-4 administration may enhance learning and memory capabilities in Alzheimer's disease patients.

### Example 7: Verification of Amyloid Plaque Formation Inhibition in an Alzheimer's Disease Animal Model

After completing the animal behavioral experiments, brain tissue sections were prepared from each animal. Thioflavin S staining was performed on these brain sections to visualize amyloid plaques. The number of stained amyloid plaques in the cortex and hippocampus was counted, and the Thioflavin S-positive regions were analyzed using the ImageJ program.

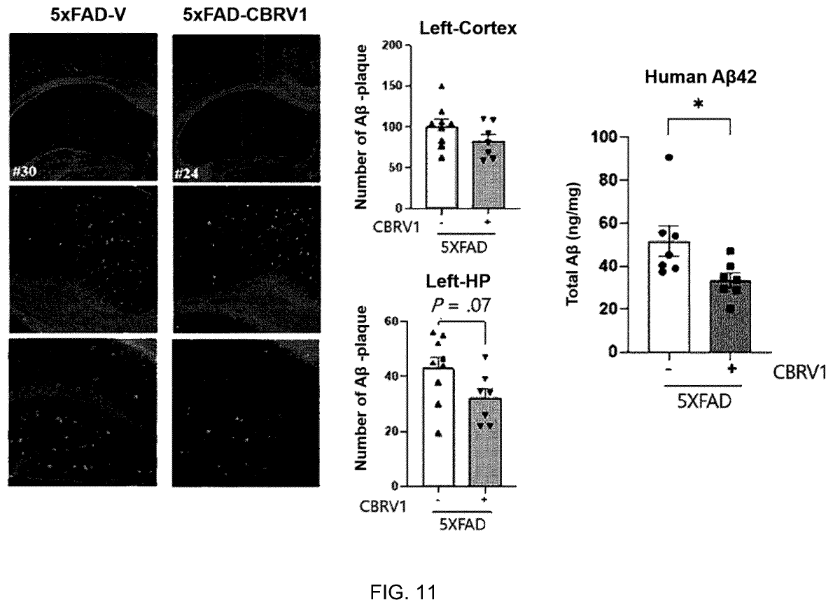

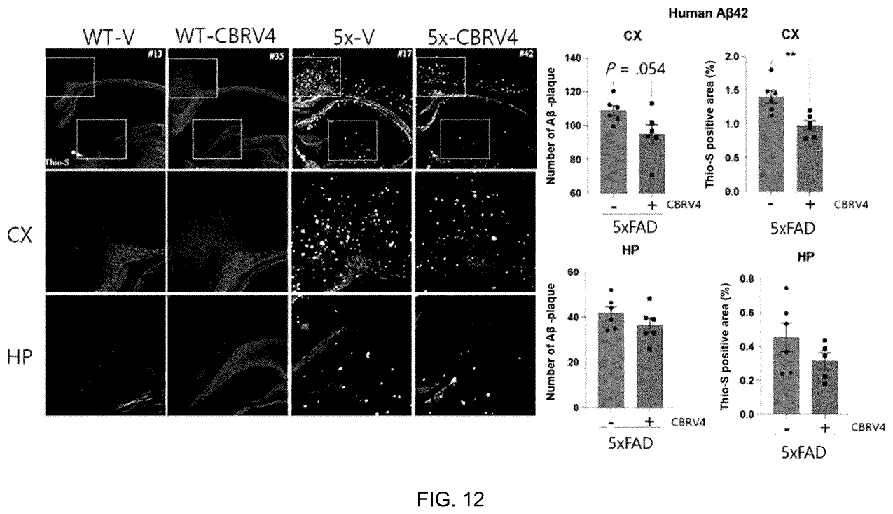

The results are shown in FIGS. 11 and 12.

In WT mice, no amyloid plaques were observed. As shown in FIG. 11, in the cortex (left), the 5×FAD-CBRV-1 treatment group exhibited a reduction in amyloid plaque numbers compared to the 5×FAD-vehicle group. In the hippocampus (HP) (left), the 5×FAD-CBRV-1 treatment group showed a statistically significant decrease in amyloid plaque numbers compared to the 5×FAD-vehicle group.

As shown in FIG. 12, in the cortex (CX) region, the 5×FAD-CBRV-4 treatment group showed a decreasing trend in amyloid plaque numbers compared to the 5×FAD-vehicle group. Measurement of Thioflavin S-positive areas indicated that both the amyloid plaque size and intensity in the cortex were significantly reduced in the 5×FAD-CBRV-4 treatment group compared to the 5×FAD-vehicle group. In the hippocampus (HP) region, the 5×FAD-CBRV-4 treatment group exhibited a decreasing trend in amyloid plaque numbers compared to the 5×FAD-vehicle group. Similarly, Thioflavin S-positive area measurements indicated that amyloid plaque size and intensity in the hippocampus were reduced in the 5×FAD-CBRV-4 treatment group compared to the 5×FAD-vehicle group.

These findings suggest that CBRV-1 and CBRV-4 administration may inhibit amyloid plaque formation in Alzheimer's disease patients.

### Example 8: Verification of Aβ Oligomerization Inhibition in an Alzheimer's Disease Animal Model

Since the MDS technique detects various oligomer forms, Western blot analysis was conducted to evaluate the inhibition of oligomer formation for specific molecular weights.

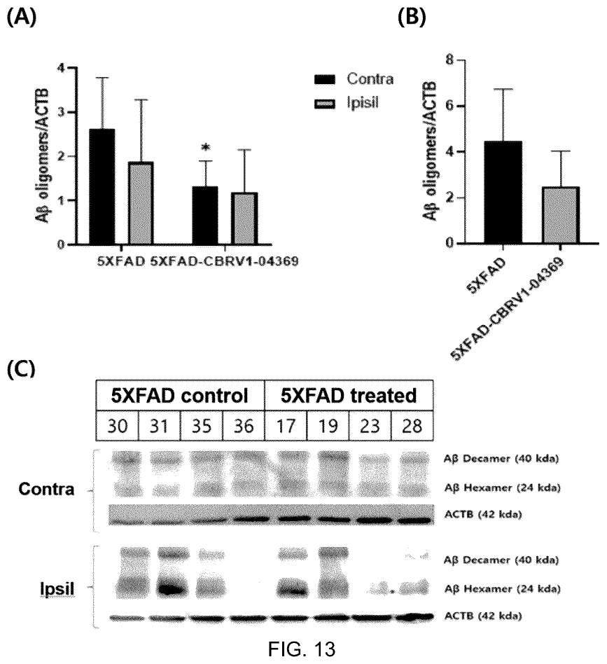

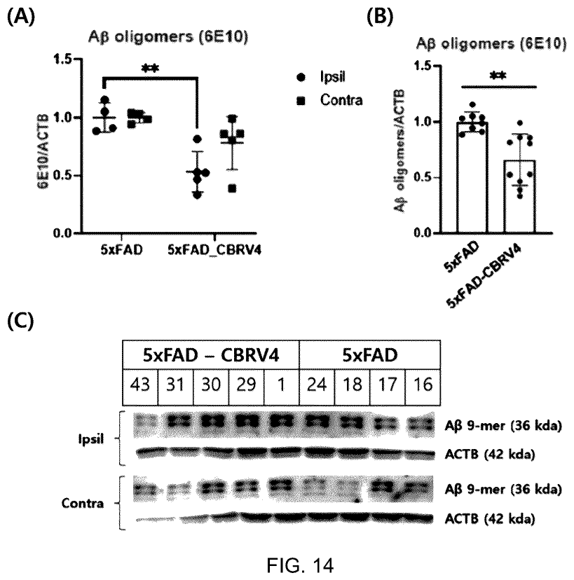

The results are shown in FIGS. 13 and 14.

As shown in FIG. 13, Western blot analysis of brain homogenates from Alzheimer's model mice confirmed the presence of Aβ oligomer hexamers (6-mer) and decamers (10-mer). After normalization with actin, the CBRV-1-treated group showed a statistically significant reduction in Aβ oligomers (Hexamer & Decamer) compared to the control.

As shown in FIG. 14, Western blot analysis using the 6E10 antibody confirmed the presence of Aβ oligomer decamers (10-mer). In the CBRV-4-treated group, the contralateral (Contralateral) side exhibited a decreasing trend in Aβ oligomer levels, while the ipsilateral (Ipsilateral) side showed a statistically significant reduction in Aβ oligomers.

### Example 9: Verification of Changes in Inflammation-Related Markers Upon Peptide Treatment

To evaluate the effect of peptide treatment on inflammation-related markers, C3 levels were measured in mouse brain homogenates and blood. C3 was measured using an ELISA kit (ab263884, Abcam) according to the manufacturer's protocol.

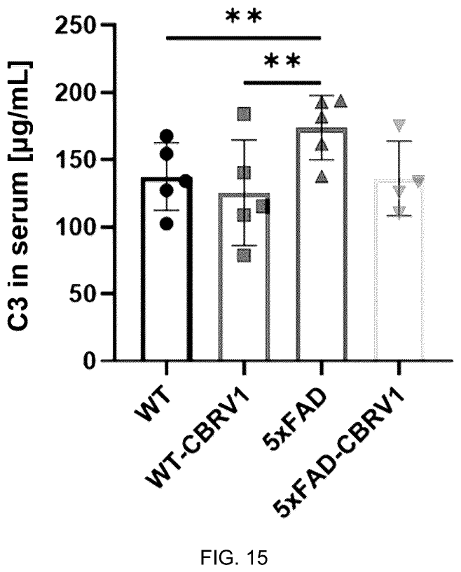

The results are shown in FIG. 15.

As illustrated in FIG. 15, C3 levels in the blood were significantly increased in the 5×FAD group compared to other groups. In the 5×FAD-CBRV-1-treated mice, although no statistically significant differences were observed, C3 levels showed a decreasing trend similar to normal mice, suggesting that CBRV-1 administration may reduce inflammation-related responses mediated by C3 in the bloodstream.

1. A polypeptide consisting of at least one selected from the group consisting of SEQ ID NOS: 3, 4, 5, 6, and 7.

2. The polypeptide of claim 1, wherein the polypeptide comprises the amino acid sequence of SEQ ID NO: 1.

3. A nucleic acid molecule comprising a nucleotide sequence encoding the polypeptide of claim 1.

4. The nucleic acid molecule of claim 3, wherein the nucleotide sequence is represented by SEQ ID NO: 2.

5. A recombinant vector comprising the nucleic acid molecule of claim 3.

6. A host cell comprising the recombinant vector of claim 5.

7. A pharmaceutical composition for treating amyloid beta-associated neurodegenerative disease, comprising the polypeptide of claim 1.

8. The pharmaceutical composition of claim 7, wherein the polypeptide comprises the amino acid sequence of SEQ ID NO: 1.

9. The pharmaceutical composition of claim 7, wherein the polypeptide inhibits fibrillation and oligomerization of amyloid beta.

10. The pharmaceutical composition of claim 7, wherein the neurodegenerative disease is any one selected from the group consisting of Alzheimer's disease, amyloid angiopathy, amyloid stroke, systemic amyloidosis, and Parkinson's disease.

11. A composition for detecting amyloid beta protein, comprising the polypeptide of claim 1.

12. The composition of claim 11, wherein the polypeptide comprises the amino acid sequence of SEQ ID NO: 1.

13. A method for detecting amyloid beta protein, comprising:
a step of contacting a sample with the composition of claim 11 to form a peptide-amyloid beta protein complex and detecting the complex.

14. A method for treating amyloid beta-associated neurodegenerative disease, comprising:
administering an effective amount of a pharmaceutical composition comprising the polypeptide including at least one amino acid sequence selected from the group consisting of SEQ ID NOS: 3, 4, 5, 6, and 7 to a subject in need thereof.

15. The method of claim 14, wherein the polypeptide comprises the amino acid sequence of SEQ ID NO: 1.

16. The method of claim 14, wherein the neurodegenerative disease is any one selected from the group consisting of Alzheimer's disease, amyloid angiopathy, amyloid stroke, systemic amyloidosis, and Parkinson's disease.
